(19) United States
(12) Patent Application Publication
(10) Pub. No.: US 2011/0284002 A1
(43) Pub. Date: Nov. 24, 2011

(54) PNEUMATIC TRANSIENT HANDLER AND METHOD

(75) Inventor: Göran Cewers, Limhamn (SE)

(73) Assignee: MINDRAY MEDICAL SWEDEN AB, Sundbyberg (SE)

(21) Appl. No.: 13/109,605

(22) Filed: May 17, 2011

Related U.S. Application Data
(60) Provisional application No. 61/345,825, filed on May 18, 2010.

(51) Int. Cl. A61M 16/00

(52) U.S. Cl. 128/204.21

(57) ABSTRACT

A method and device for handling transient pressures and flow processes in a breathing system is disclosed.

### CROSS-REFERENCE TO RELATED APPLICATION(S)

This application claims the benefit of U.S. Provisional Application No. 61/345,825, filed May 18, 2010, which is incorporated herein by reference.

### TECHNICAL FIELD

The following disclosure relates to breathing systems.

### SUMMARY OF THE INVENTION

A method and device for controlling or compensating for transient pressures and flow processes in a breathing system is disclosed.

### BRIEF DESCRIPTION OF THE DRAWINGS

FIG. 1 is a schematic view of a standard breathing system configuration;

FIG. 2 is a schematic view of a breathing system being supplemented with a pressure compensation device; and

FIG. 3 is a schematic view of a pressure compensation device.

### DETAILED DESCRIPTION

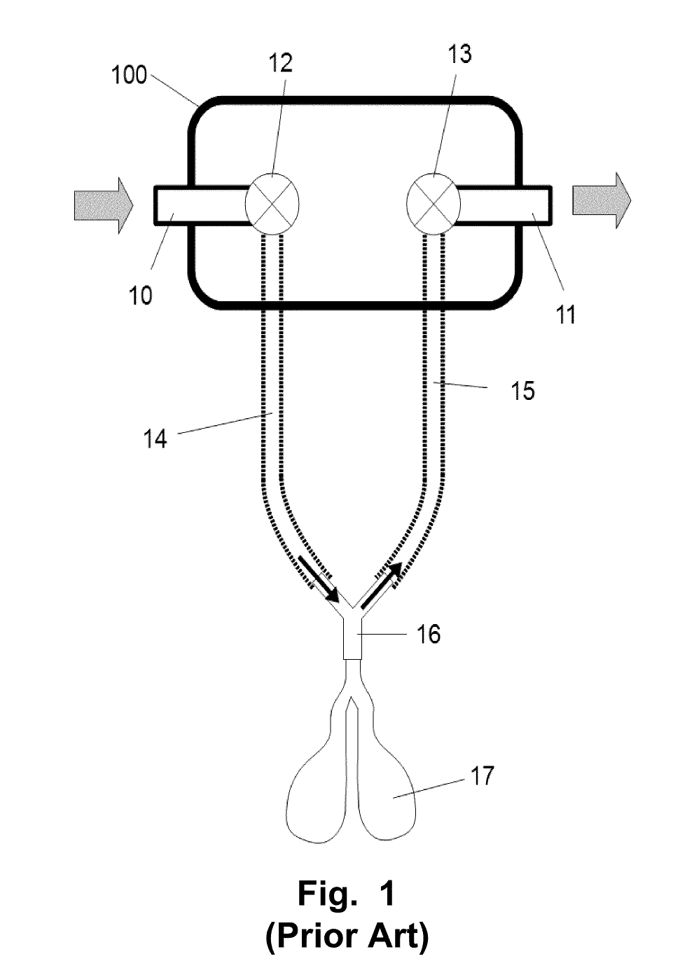

One of the most common breathing system configurations in medical ventilators today is illustrated in FIG. 1. A ventilator 100 has the primary task of alternatingly delivering a quantity of gas into the patient's lung 17, and to subsequently release the delivered volume.

The gas flows are usually controlled by valves 12 and 13, which are connected to tubes 14 and 15, which feed together into a Y-piece 16, and then flow through a tube or mask to the patient. Since the valves with a control system and a power supply are relatively bulky, they are usually assembled in a housing, and collectively constitute the ventilator. Also illustrated are an inlet 10 connected to the value 12 and an outlet connected to the valve 13, as well as an inlet/outlet 21 connecting a pressure compensation device 20 with the Y-piece 16 (shown) or the ventilator tubes.

The ventilator is generally placed a few meters from the patient. Consequently, the ventilator tubes are often a meter or more in length. This is not an insuperable issue in terms of dosing, as one tube is always filled with inhalation gas and the other with exhalation gas, and new gas being supplied to the patient merely displaces the gas volume, which is already present in the tubes without any major faults in volume, as induced pressure changes do not result in any serious volume changes.

Pressure changes are induced into the tubes when the flows change. The weight of the gas mass in the tubes, in combination with the gas's compressibility, cause harmonic oscillations in the tube system. The patient may experience discomfort from these oscillations.

The pressure changes also cause problems in pressure controlled respiratory modes. This is because pressure control is usually obtained by means of a so-called “closed loop” technology. In order for closed loop control to be performed distinctly and accurately, the loop must be kept short. This can be done by positioning the pressure sensor in the loop close to the valve, which controls the gas that is to be pressure controlled. Unfortunately, the pressure profile will be different at the Y-piece from that at the pressure sensor. If, instead, one attempts to place the pressure sensor at the Y-piece, the pressure regulation loop becomes large, with the consequent impaired pressure control. Yet another cause of the undesirable pressure profile at the Y-piece may be that the gas source control is slow, which is often the case if the gas source consists of a flow variable turbine, or if the inspiration valve is slow.

According to the disclosure, a flow profile is generated at the Y-piece or a breathing mask that corrects the pressure to a desired pressure profile. This can be done by using an active element that generates a flow, compensating for undesirable pressure. A device of this kind can generate a flow profile that is not possible to obtain from a distant positioned inspiration valve. For example, a method may be implemented in HFO or HFV applications.

According to one embodiment of the disclosure, a flow generating device is connected in the gas channel close to the patient. The flow generated in this way should be bi-directional and have a broad bandwidth, but may have a mean value of zero, as a mean value different than zero can be generated by the inspiration valve. In principle, the flow generating device may comprise a loudspeaker element, but a specific design adapted to the application may be used.

An alternative to an ordinary loudspeaker is to replace the coil with a piezoelectric actuator combined with a mechanical amplifier. This alternative will considerably improve the bandwidth of the device.

The pressure is measured close to the patient in order to properly control the device flow, resulting in a pressure signal. This pressure signal is included in another pressure control loop, whose desired control value is the desired pressure profile at the patient, and whose output signal controls the device actuator.

Alternatively, the device may also be used as a stand alone unit next to the patient. The device may then help to actively counteract oscillations, according to predetermined criteria.

According to one aspect of the disclosure, a pneumatic transient pressure handler unit is provided for connection to, and control of, pressure in a breathing system. The transient pressure handler may include at least one actuator element, at least one container with an outlet channel, and at least one pressure sensor. The movement of the actuator element compresses or decompresses the container, depending on the pressure measured by the pressure sensor. The container's outlet channel may be connected to the breathing system, wherein the transient pressure handler compensates for undesired pressure variations in the breathing system.

As a result, the pneumatic transient pressure handler may be connected to a flow generating device, such as a respirator system, and there act as an active element that is both bi-directional and is broad in bandwidth. The desired pressure profiles may be created by controlling the system with a pressure control loop, which, in turn, is connected to another pressure control loop where the pressure is measured close to the patient in a suitable manner.

The container may be configured as a bellows with a disk pressing on it, which is set in motion by the actuator.

The system may be configured as a stand-alone unit at a patient and then be used to actively counteract oscillations, according to predetermined criteria.

In one embodiment of the pneumatic transient pressure handler, actuator element 30 comprises piezo actuators. This design improves the bandwidth of the device compared with, e.g., a coil, which may also be used in other embodiments. Piezo actuators may be connected in series with a mechanical motion amplifier 31 to amplify the amplitude of a pushing or pulling movement of the piezo actuator.

In another aspect, the disclosure includes a method for controlling or compensating for transient pressures and flow processes in a breathing system. The method may include providing a pneumatic transient pressure handler connected to a breathing system to control the movement of an actuator unit in a pressure control loop of at least one pressure sensor, and by means of the motion, create a flow in and out of the pneumatic transient pressure handler unit to generate a desired pressure profile in the breathing system.

Accordingly, the disclosed method may produce a desired pressure profile at the patient, e.g., remove pressure oscillations, which are induced in the breathing system tubes by pressure changes and which can give rise to harmonic oscillations. The patient may experience discomfort from harmonic oscillations, which may cause problems with pressure controlled respiration methods.

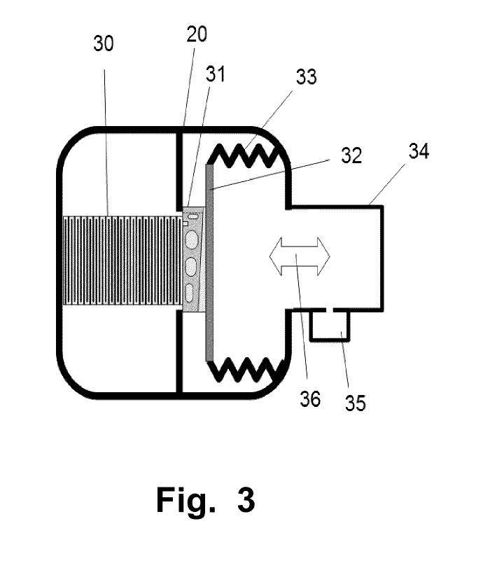

FIG. 3 shows, in a schematic view, an exemplary embodiment of the pressure compensation device 20 with a piezoelectric actuator 30, a mechanical amplifier 31, a movable disk 32, a bellows 33, and an outlet 34. The bi-directional arrow 36 shows the direction of the bi-directionally controlled flow.

An example of a device according to the disclosure may be obtained, as shown in FIG. 3, in that the motion from a piezoelectric actuator 30 is amplified by means of a mechanical amplifier 31, which, in turn, brings disk 32 into motion. Bellows 33 is then compressed and a momentary flow 36 is then created through outlet 34. Both actuator 30 and movable disk 32 may be spring-loaded (not shown in the figures) so that movements and flows can be generated both ways. A pressure sensor 35 may provide feedback into the pressure control loop for the device.

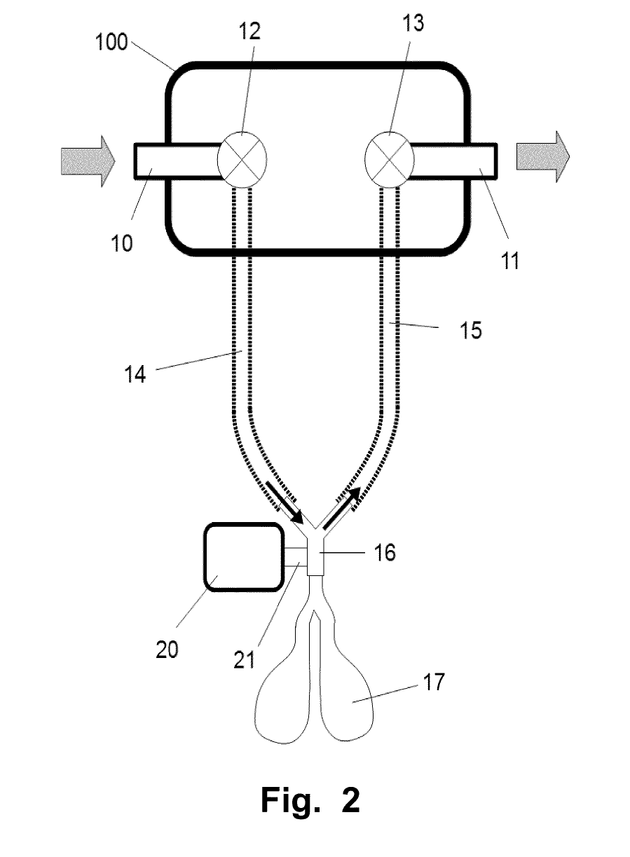

FIG. 2 shows how the breathing system can be supplemented with a pressure compensation device 20. The connection to the breathing system is shown close to the patient, e.g., at the Y-piece.

Without further elaboration, it is believed that one skilled in the art can use the preceding description to utilize the present disclosure to its fullest extent. The examples and embodiments disclosed herein are to be construed as merely illustrative and not a limitation of the scope of the present disclosure in any way. It will be apparent to those having skill in the art that changes may be made to the details of the above-described embodiments without departing from the underlying principles of the disclosure described herein. In other words, various modifications and improvements of the embodiments specifically disclosed in the description above are within the scope of the appended claims. The scope of the invention is, therefore, defined by the following claims. The words “including” and “having,” as used herein, including the claims, shall have the same meaning as the word “comprising.”

What is claimed is:

1. A pneumatic transient handler unit for connection to a breathing system to control pressure within the breathing system, comprising:
at least one actuator element;
at least one container with an outlet channel; and
at least one pressure sensor;
wherein a motion of the actuator element compresses or decompresses the container depending on the pressure measured by the pressure sensor, and
wherein the outlet channel of the container is connected to the breathing system, whereby the pneumatic transient handler unit compensates for undesired pressure variations in the breathing system.

2. The transient handler unit according to claim 1, wherein the pneumatic transient handler is arranged and controlled to contribute to maintain a desired pressure profile in the breathing system.

3. The transient handler unit according to claim 1, wherein the pneumatic transient handler is configured for stand alone operation with pressure compensation based on predetermined criteria.

4. The transient handler unit according to claim 1, wherein the actuator element comprises at least one piezo actuator.

5. The transient handler unit according to claim 4, wherein a mechanical motion amplifier is serially coupled to the actuator element.

6. The transient handler unit according to claim 1, wherein said container comprises a bellows.

7. The transient handler unit according to claim 6, wherein the actuator element is arranged to bring a disk in motion for changing a volume of said bellows.

8. A method of controlling or compensating for transient pressures and flow processes in a breathing system, the method comprising:
providing a pneumatic transient handler unit coupled to a breathing system;
controlling a motion of said actuator unit in a pressure control loop using at least one pressure sensor; and
generating a flow into and out of said pneumatic transient handler unit for generating a desired pressure profile in the breathing system.

9. The method according to claim 8, wherein said pneumatic transient handler unit is arranged close to a patient who is connected to said breathing system.
